SEQUENCE LISTING (13 sequences)

SEQ ID NO: 1 - protein, 32 aa, Synthetic construct
  CROSSLNK 1..7: note = Bridge between location 1 (X1) and location 7 (X7)
  CROSSLNK 11..22: note = Bridge between location 11 (Lys) and location 22 (Tyr)
  VARIANT 18: note = X=Lys or modified Lys comprising a bulky moiety connected to its epsilon-amine
  VARIANT 1: note = X=Cys or modified Cys
  VARIANT 7: note = X=Cys or modified Cys
  VARIANT 24: note = X=Arg or Gln
XSNLSTXVLGKLSQELHXLQTYPXTNTGSGTP

SEQ ID NO: 2 - protein, 32 aa, Synthetic construct
  DISULFID 1..7: note = Disulfide bridge between location 1 (Cys) and location 7 (Cys)
  CROSSLNK 11..22: note = Methylene bridge between location 11 (Lys) and location 22 (Tyr)
  MOD_RES 32: note = location 32 (Pro) is amidated
  VARIANT 24: note = X=Arg or Gln
CSNLSTCVLGKLSQELHKLQTYPXTNTGSGTP

SEQ ID NO: 3 - protein, 32 aa, Synthetic construct
  DISULFID 1..7: note = Disulfide bridge between location 1 (Cys) and location 7 (Cys)
  CROSSLNK 11..22: note = Methylene bridge between location 11 (Lys) and location 22 (Tyr)
  MOD_RES 32: note = Location 32 (Pro) is amidated
CSNLSTCVLGKLSQELHKLQTYPRTNTGSGTP

SEQ ID NO: 4 - protein, 32 aa, Synthetic construct
  DISULFID 1..7: note = Disulfide bond between location 1 (Cys) and location 7 (Cys)
  MOD_RES 32: note = Location 32 (Pro) is amidated
  CROSSLNK 11..22: note = Methylene bridge between location 11 (Lys) and location 22 (Tyr)
CSNLSTCVLGKLSQELHKLQTYPQTNTGSGTP

SEQ ID NO: 5 - protein, 32 aa, Oncorhynchus keta; identical to SEQ ID NO: 3
  DISULFID 1..7: note = Disulfide bond between location 1 (Cys) and location 7 (Cys)
  MOD_RES 32: note = Location 32 (Pro) is amidated

SEQ ID NO: 6 - protein, 32 aa, Homo sapiens
  DISULFID 1..7: note = Disulfide bond between location 1 (Cys) and location 7 (Cys)
  MOD_RES 32: note = Location 32 (Pro) is amidated
CGNLSTCMLGTYTQDFNKFHTFPQTAIGVGAP

SEQ ID NO: 7 - protein, 32 aa, Synthetic construct; identical to SEQ ID NO: 3
  CROSSLNK 1..7: note = Methylene thioacetal bridge between location 1 (Cys) and location 7 (Cys)
  MOD_RES 32: note = Location 32 (Pro) is amidated

SEQ ID NO: 8 - protein, 32 aa, Synthetic construct
  CROSSLNK 1..7: note = Diselenide bridge between location 1 (Sec, U) and location 7 (Sec, U)
  MOD_RES 32: note = Location 32 (Pro) is amidated
USNLSTUVLGKLSQELHKLQTYPRTNTGSGTP

SEQ ID NO: 9 - protein, 32 aa, Synthetic construct; identical to SEQ ID NO: 3
  DISULFID 1..7: note = Disulfide bridge between location 1 (Cys) and location 7 (Cys)
  CROSSLNK 15..18: note = Lactam bridge between location 15 (Glu) and location 18 (Lys)
  MOD_RES 32: note = Location 32 (Pro) is amidated

SEQ ID NO: 10 - protein, 32 aa, Synthetic construct; identical to SEQ ID NO: 3
  DISULFID 1..7: note = Disulfide bridge between location 1 (Cys) and location 7 (Cys)
  CROSSLNK 18..22: note = Methylene bridge between location 18 (Lys) and location 22 (Tyr)
  MOD_RES 32: note = Location 32 (Pro) is amidated

SEQ ID NO: 11 - protein, 32 aa, Synthetic construct
  DISULFID 1..7: note = Disulfide bridge between location 1 (Cys) and location 7 (Cys)
  CROSSLNK 11..22: note = Methylene bridge between location 11 (Lys) and location 22 (Tyr)
  VARIANT 18: note = X=Lys or modified Lys comprising a bulky moiety connected to its epsilon-amine
  MOD_RES 32: note = Location 32 (Pro) is amidated
CSNLSTCVLGKLSQELHXLQTYPQTNTGSGTP

SEQ ID NO: 12 - protein, 32 aa, Synthetic construct
  CROSSLNK 1..7: note = Bridge between location 1 (X1) and location 7 (X7)
  VARIANT 1: note = X= Cys or modified Cys
  VARIANT 7: note = X=Cys or modified Cys
  MOD_RES 32: note = Location 32 (Pro) is amidated
  CROSSLNK 11..22: note = Methylene bridge between location 11 (Lys) and location 22 (Tyr)
XSNLSTXVLGKLSQELHKLQTYPQTNTGSGTP

SEQ ID NO: 13 - protein, 32 aa, Synthetic construct
  CROSSLNK 1..7: note = Bridge between location 1 (X1) and location 7 (X7)
  CROSSLNK 11..22: note = Methylene bridge between location 11 (Lys) and location 22 (Tyr)
  VARIANT 1: note = X=Cys or modified Cys
  VARIANT 7: note = X=Cys or modified Cys
  VARIANT 18: note = X=Lys or modified Lys comprising a bulky moiety connected to its epsilon-amine
  MOD_RES 32: note = Location 32 (Pro) is amidated
XSNLSTXVLGKLSQELHXLQTYPQTNTGSGTP

(19) United States
(12) Patent Application Publication
(10) Pub. No.: US 2026/0308222 A1
(43) Pub. Date: Oct. 8, 2026

(54) STABILIZED ANALOGUES OF CALCITONIN

(71) Applicant: YISSUM RESEARCH DEVELOPMENT COMPANY OF THE HEBREW UNIVERSITY OF JERUSALEM LTD., Jerusalem (IL)

(72) Inventors: Norman MET ANIS, Jerusalem (IL); Hiba GHAREEB, Jerusalem (IL)

(21) Appl. No.: 19/164,257

(22) PCT Filed: Mar. 10, 2024
(86) PCT No.: PCT/IL2024/050258

§ 371 Date: Sep. 11, 2025

(51) Int. Cl. C07K 14/585; A61K 9/00; A61K 38/00; A61K 45/06; A61P 19/10

(52) U.S. Cl. CPC C07K 14/585; A61K 9/0019; A61K 9/0053; A61K 45/06; A61P 19/10; A61K 38/00

(57) ABSTRACT

The present invention provides stabilized calcitonin analogues that demonstrate improved stability and bioavailability. The present invention also provides pharmaceutical compositions of the stabilized calcitonin analogues as well as their use in methods of treating and preventing calcium-related and bone-related disorders and diseases.

### FIELD OF THE INVENTION

The present invention is in the fields of peptide chemistry, calcium homeostasis and bone biology and remodeling and relates to stabilized calcitonin analogues, pharmaceutical formulations, and methods of use thereof.

### BACKGROUND OF THE INVENTION

Human calcitonin (hCT) is an endogenous peptide hormone secreted by the parafollicular C cells of the thyroid gland that plays a critical role in regulating calcium homeostasis and bone remodeling (Copp, 1970). hCT is formed by cleavage of the pre-peptide product of the CALC1 gene to form a 32 amino acids peptide cyclized by a disulfide bridge formed between two Cysteine (Cys, C) residues at positions 1 and 7. The function of hCT is derives from its ability to inhibit bone resorption, and reduce osteoclast formation and attachment (Reginster, 1993). Calcitonin (CT) is found in various organisms including mammals and fish (Azria et al., 1995) and shares several structural features across species, including an N-terminal disulfide bridge (between Cys1 and Cys7) and a proline amide at the C-terminus. Folding into a helical structure is also a structural feature of CT across species and is essential for receptor binding (Andreotti et al., 2006).

The most widely used CT variant in therapy is salmon calcitonin (sCT), also known as salcatonin, since it has the highest affinity for the human calcitonin receptor (hCTR), and the highest potency (Potts Jr., 1992) while human hCT is an aggregation-prone peptide known to form amyloid fibrils (Reches, et al., 2002; Iconomidou, et al. 2013). Hence, sCT has been used for more than 40 years to treat postmenopausal osteoporosis, Paget's disease and hypercalcemia (Azria et al., 1995). In addition, calcitonin reduces the risk of vertebral fracture recurrence and provides pain relief for acute back pain caused by vertebral fractures (Chesnut III, et al., 2000) and is commercially available as an injection and nasal spray. The intra-muscular and subcutaneous injections of sCT are available as parenteral calcitonin dosage forms, which are associated with gastrointestinal (GI) disturbances and injection site reactions (Wimalawansa, 1993; Wuster et al, 1991). Intranasal administration by nasal spray has many advantages over the parenteral form, such as convenience and less invasive for patient, and fewer GI side effects. However, the nasal spray has lower bioavailability and slower absorption compared to the parenteral (Srinivasan et al., 2020).

Enhancing the stability of therapeutic peptides such as sCT in the GI tract would improve their therapeutic application. Oral administration is the most convenient route for drug administration, due to its cost effectiveness, ease of ingestion, painlessness, and high patient compliance. However, peptides are vulnerable to enzymatic degradation in the GI tract, lowering their efficacy in patients to unacceptable levels (Hamman et al., 2005). One of the main obstacles to the development of oral CT is its degradation in the acidic environment of gastric fluid and the proteases found in the stomach and the intestine, including pepsin, trypsin, chymotrypsin and elastase (Wang et al., 2015). Several oral formulations have been developed to prevent sCT degradation (Karsdal et al., 2010), however, they failed phase III clinical trials due to insignificant effect in reducing the risk of vertebral and non-vertebral fractures (Karsdal et al., 2015).

Many synthetic approaches can be utilized to stabilize peptides including helical peptide stapling, in which crosslinking between side chains constrain the peptide's molecular flexibility, usually improving binding affinity, selectivity and stability (Verdine and Walensky, 2007; Bock, et al., 2013; Schafmeister, et al. 2000; Lau, et al., 2014). Macrocyclic or helix stapled peptides are known to have higher resistance to protease degradation as well as enhanced potency and binding affinity (Walensky and Bird, 2014; Charoenpattarapreeda, et al., 2019; Cromm, et al., 2015). Examples of crosslinking approaches include helix stabilization, such as lactam groups, disulfide mimics, hydrocarbon stapled peptides, and others (Phelan, et al., 1997; Bracken, et al., 1994; Lau, et al., 2015; Chang et al., 2013). More recently, a new type of stapling of linear peptides was reported (Li, et al., 2021; Li, et al., 2022). In this method, formaldehyde forms an imine with the s-amino group of a lysine (Lys, K) residue, followed by crosslinking to a tyrosine (Tyr, Y) residue in the polypeptide chain.

U.S. Pat. No. 6,107,277 discloses modified active calcitonin analogues in which the disulfide bridge between Cys1 and the Cys7 is broken and the two cysteine molecules are derivatized with various substituents.

WO2009/019307 discloses pharmaceutical compositions of CT comprising a specific oral delivery agent and specific amounts of vitamin D and a calcium salt.

There remains an unmet need for bioavailable and metabolically stable calcitonin analogues for the treatment of calcium-related diseases.

### SUMMARY OF THE INVENTION

The present invention provides stapled calcitonin (CT) analogues, in particular metabolically stable and orally available analogues. The stapled CT analogues of the present invention are conformationally stabilized by two bridges that constrain their helical conformation. The invention is based on the unexpected discovery that CT analogues that are conformationally stabilized and comprise specific sequence modifications demonstrate superior stability as compared to CT molecules currently used therapeutically. These novel CT analogues are more biologically available and may be used for prevention and treatment of calcium-related and bone-related disorders, advantageously by oral administration.

The present invention provides according to one aspect, a stabilized, bicyclic peptide analogue of calcitonin (CT) comprising the amino acid sequence X₁-Ser-Asn-Leu-Ser-Thr-X₂-Val-Leu-Gly-Lys-Leu-Ser-Gln-Glu-Leu-His-X₃-Leu-Gln-Thr-Tyr-Pro-X₄-Thr-Asn-Thr-Gly-Ser-Gly-Thr-Pro (SEQ ID NO: 1), wherein X₁ and X₂ are independently selected from Cys (C), and modified Cys residues; X₃ is selected from Lys (K) and a modified Lys residue comprising a bulky moiety connected to its F-amine; and X₄ is selected from Gln (Q) and Arg (R), wherein a first bridge connects positions 1 and 7 and a second bridge connects positions Lys11 and Tyr22; or a derivative thereof comprising 1-3 modifications to SEQ ID NO: 1.

According to some embodiments, the 1-3 modifications are selected from substitutions, additions, or deletions of amino acids, modification of at least one of the termini of the peptide, and combinations thereof.

According to some embodiments, the bicyclic peptide analogue consists of 29-35 amino acids.

According to some embodiments, the bicyclic peptide analogue consists of SEQ ID NO: 1 with an amidated C-terminus.

According to some embodiments, X₁ and X₂ are each independently selected from a Cys (C) and a Sec (U) residue.

According to some embodiments, X₁ and X₂ are each a Cys residue.

According to some embodiment, the first bridge between Cys1 and Cys7, is a disulfide bridge.

According to some embodiments, X₁ and X₂ in each position is a modified Cys residue independently selected from: selenocysteine (Sec, U) and Sec-SeH, Cys-S—H, Cys-OH, Cys-S—OH, Cys-S—O—(CH₂)₁₋₄—CH₃, Cys-S-ester-(CH₂)₁₋₄—CH₃, and Cys-S-acetamidomethyl.

According to some embodiments, the N-terminus of the peptide is acetylated.

According to some embodiments, the N-terminal residue of the peptide is an acetylated Cys residue.

According to some embodiments, the peptide analogue comprises an amidated C-terminus. According to some specific embodiments, the C-terminal residue of the peptide is Pro-NH₂.

According to some embodiments, X₃ is Lys (K).

According to some embodiments, X₃ is a modified Lys residue comprising a bulky moiety connected to its ε-amine.

According to some embodiments, the bulky moiety is selected from a polyethylene glycol (PEG) moiety and a fatty acid moiety.

According to some specific embodiments, the PEG moiety comprises PEG₈₋₂₀.

According to some specific embodiments, the PEG moiety comprises PEG₁₀₋₁₄.

According to some embodiments, the second bridge, between Lys11 and Tyr22, is an alkylene bridge comprising 1-6 methylene units, or a modified alkylene bridge.

According to some embodiments, the second bridge is selected from a methylene bridge, an ethylene bridge, and a propylene bridge.

According to some embodiments, the second bridge is a methylene bridge.

According to some embodiments, the second bridge is a modified alkylene bridge.

According to some embodiments, the modified alkylene bridge is selected from (—CH₂—), (—CH₂—O—CH₂—), (—CF₂—), (—CH₂CH₂—O—CH₂CH₂—), (—CF₂CF₂—) and similar.

According to some embodiments, X₄ is Arg (R).

According to some embodiments, X₄ is Gln (Q).

According to some embodiments, the bicyclic calcitonin analogue comprises the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-X₄-Thr-Asn-Thr-Gly-Ser-Gly-Thr-Pro-NH₂ (SEQ ID NO: 2), wherein X₄ is selected from Q and R, or a derivative comprising 1-3 modifications.

According to some embodiments, the stabilized, bicyclic peptide analogue consists of a sequence selected from:
Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Gln-Thr-Asn-Thr-Gly-Ser-Gly-Thr-Pro-NH₂, wherein a disulfide bridge connects residues Cys1 and Cys7, and a methylene bridge connects residues Lys11 and Tyr22 (SEQ ID NO: 4, designated KaY-1(R24Q));
Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Asn-Thr-Gly-Ser-Gly-Thr-Pro-NH₂, wherein a disulfide bridge connects residues Cys1 and Cys7, and a methylene bridge connects residues Lys11 and Tyr22 (SEQ ID NO: 3, designated KaY-1);
Cys-Ser-Asn-Leu-Ser-Thr-Cys-Val-Leu-Gly-Lys-Leu-Ser-Gln-Glu-Leu-His-X₃-Leu-Gln-Thr-Tyr-Pro-Gln-Thr-Asn-Thr-Gly-Ser-Gly-Thr-Pro-NH₂, wherein a disulfide bridge connects residues Cys1 and Cys7, a methylene bridge connects residues Lys11 and Tyr22, and X₃ is selected from Lys or modified Lys comprising a bulky moiety connected to its ε-amine (SEQ ID NO: 11, designated pegylated KaY-1(R24Q));
X₁-Ser-Asn-Leu-Ser-Thr-X₂-Val-Leu-Gly-Lys-Leu-Ser-Gln-Glu-Leu-His-Lys-Leu-Gln-Thr-Tyr-Pro-Gln-Thr-Asn-Thr-Gly-Ser-Gly-Thr-Pro-NH₂, wherein X₁ and X₂ are independently selected from Cys (C) and modified Cys residues, and wherein a bridge connects residues X₁ and X₂ and a methylene bridge connects residues Lys11 and Tyr22 (SEQ ID NO: 12, designated Cys-modified KaY-1(R24Q));
X₁-Ser-Asn-Leu-Ser-Thr-X₇-Val-Leu-Gly-Lys-Leu-Ser-Gln-Glu-Leu-His-X₃-Leu-Gln-Thr-Tyr-Pro-Gln-Thr-Asn-Thr-Gly-Ser-Gly-Thr-Pro-NH₂, wherein X₁ and X₂ are independently selected from Cys (C) and modified Cys residues and wherein a bridge connects residues X₁ and X₂, a methylene bridge connects residues Lys11 and Tyr22, and X₃ is selected from Lys or modified Lys comprising a bulky moiety connected to its ε-amine (SEQ ID NO: 13, designated Cys-modified pegylated KaY-1(R24Q)).

According to some embodiments, the bicyclic calcitonin analogue consists of the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-X₄-Thr-Asn-Thr-Gly-Ser-Gly-Thr-Pro-NH₂ (SEQ ID NO: 2), wherein X₄ is selected from Q and R, wherein a disulfide bridge connects residues Cys1 and Cys7, and a methylene bridge connects residues Lys11 and Tyr22.

According to some embodiments, the bicyclic calcitonin analogue consists of the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Gln-Thr-Asn-Thr-Gly-Ser-Gly-Thr-Pro-NH₂, wherein a disulfide bridge connects residues Cys1 and Cys7, and a methylene bridge connects residues Lys11 and Tyr22 (SEQ ID NO: 4, designated KaY-1(R24Q)).

According to some embodiments, the bicyclic calcitonin analogue consists of the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Asn-Thr-Gly-Ser-Gly-Thr-Pro-NH₂, wherein a disulfide bridge connects residues Cys1 and Cys7, and a methylene bridge connects residues Lys11 and Tyr22 (SEQ ID NO: 3, designated KaY-1).

The present invention also provides, according to yet another aspect, a conjugate comprising a CT analogue having a sequence set forth in any one of SEQ ID NOs: 1-4 and 11-13, or a derivative thereof comprising 1-3 modifications to any of these sequences, and at least one additional moiety or sequences connected to the CT analogue or derivative.

According to some embodiments, 1-15 amino acids are connected to the CT analogue or derivative to form a conjugate consisting of 30-50, 30-45, 30-40, or 30-35 amino acids.

According to some embodiments, the conjugate comprises at least one non-proteinaceous moiety.

According to some embodiments, the conjugate comprises one or more permeability-enhancing moieties.

According to some embodiments, the conjugate comprises one or more stabilizing moieties.

According to some aspects and embodiments, the present invention provides a pharmaceutical composition comprising a stabilized bicyclic calcitonin analogue according to any one of SEQ ID NOs: 1-4 and 11-13, a derivative thereof comprising 1-3 modifications, or a conjugate thereof, and a pharmaceutically acceptable carrier.

According to some embodiments, the pharmaceutical composition is formulated in a form suitable for oral, nasal, intramuscular, subcutaneous or intravenous administration. According to particular embodiments, the pharmaceutical composition is formulated in a form suitable for nasal or oral administration. According to more particular embodiments, the pharmaceutical composition is formulated in a form suitable for oral administration.

According to yet other embodiments, the pharmaceutical composition is for use in preventing or treating a calcium-related or a bone-related disorder, disease or disease complication or any disorder, disease or complication known to be responsive to the action of calcitonin.

According to some particular embodiments, the disorder is a bone disorder.

According to specific embodiments, the bone disorder is a disorder caused by bone resorption including hypercalcemia, osteoporosis (including postmenopausal osteoporosis), osteolysis, Paget's disease, or a disorder characterized by arthritic conditions including rheumatoid arthritis (RA), inflammation of the joints, swelling of skeletal joints, cartilage erosion, bone erosion, bone fractures, osteoarthritis, and bone destruction.

According to some embodiments, the disorder is selected from postmenopausal osteoporosis, Paget disease of bone, hypercalcemia, acute bone loss due to sudden immobilization and migraines. In particular embodiments, the disorder is selected from postmenopausal osteoporosis, Paget disease of bone, and hypercalcemia.

According to some embodiments, the pharmaceutical composition is for use in the treatment of pain, or for decreasing or alleviating pain associated with a bone-related or a calcium-related disorder, disease or complication in a subject.

Pharmaceutical compositions comprising a stabilized bicyclic calcitonin analogues, derivatives or conjugates described above, at least one pharmaceutically acceptable carrier and at least one additional medicament are also included within the scope of the present invention. The at least one additional medicament may include, but is not limited to, vitamin D, a calcium salt, bisphosphonates, loop diuretics, oral phosphate, corticosteroids, and non-steroidal anti-inflammatory drugs.

The present invention provides according to some further aspects and embodiments, a method of treating a calcium-related or a bone-related disorder, disease or disease complication, or any disease, disorder or complication responsive to the action of calcitonin, the method comprising providing an effective amount of a stabilized bicyclic calcitonin analogue according to any one of SEQ ID NOs: 1-4 and 11-13, a derivative thereof comprising 1-3 modifications, or a conjugate thereof, to a patient in need of such a treatment.

According to some particular embodiments, the disorder is a bone disorder. According to specific embodiments, the bone disorder is a disorder caused by bone resorption including hypercalcemia, osteoporosis (including postmenopausal osteoporosis), osteolysis, Paget's disease, or a disorder characterized by arthritic conditions including rheumatoid arthritis, inflammation of the joints, swelling of skeletal joints, cartilage erosion, bone erosion, bone fractures, osteoarthritis, and bone destruction.

According to some embodiments, the disorder is selected from postmenopausal osteoporosis, Paget disease of bone, hypercalcemia, acute bone loss due to sudden immobilization and migraines. In particular embodiments, the disorder is selected postmenopausal osteoporosis, Paget disease of bone, and/or hypercalcemia.

In some embodiments, the method further comprises providing the stabilized calcitonin with at least one other treatment or medicament. According to some embodiments, the at least one other medicament is selected from bisphosphonates, loop diuretics, oral phosphate, corticosteroids, and non-steroidal anti-inflammatory drugs.

According to some embodiments, the disorder or complication is pain.

According to some embodiments, the present invention provides a method of treating or alleviating pain, the method comprising providing an effective amount of stabilized CT according to any one of SEQ ID NOs: 1-4 and 11-13, a derivative thereof comprising 1-3 modifications, or a conjugate thereof, to a patient in need of such a treatment.

Any administration route may be used in the method of treating or alleviating a disorder, disease, or complication. Administration routes include parenteral, enteral, and topical routes.

According to some embodiments, the method of treating or alleviating a disorder, disease or complication comprises administering the stabilized CT analogue, or a pharmaceutical composition comprising said analogue, by an administration route selected from oral and nasal.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. shows NMR-derived structure of sCT (PDB entry 2GLG), demonstrating the high helicity of the peptide, the conserved disulfide bond between Cys1 and Cys7 and the conserved C-terminal proline amide.

FIG. 2. shows amino acid sequences of hCT and sCT along with several synthetic calcitonin analogues. Positions of amino acids that are crosslinked, both in nature and by design, are shown.

FIGS. 3A-3C. schematic representations of synthesis of stabilized calcitonin analogues. FIG. 3A. Synthesis of sCT, SeCT and MT-CT. FIG. 3B. Synthesis of CT(E₁₅-K₁₈). FIG. 3C. Synthesis of KaY-1, KaY-2, KaY-1(R24Q).

FIGS. 4A-4B. characterization of KaY-1 by high performance liquid chromatography (HPLC) and Circular dichroism (CD). FIG. 4A. HPLC analysis FIG. 4B. Circular dichroism analysis.

FIGS. 5A-5B. characterization of KaY-1(R24Q) from the starting material of sCT(R24Q). FIG. 5A. Top HPLC analysis shows two peaks, corresponding to KaY-1(R24Q) and to the unreacted sCT(R24Q) starting material aligned with the HPLC analysis of pure KaY-1(R24Q), shown at the bottom of the figure. FIG. 5B. electron spray ionization mass spectrometry (ESI-MS) of the purified product.

FIG. 6A-6B. Structure of KaY-1 (FIG. 6A) and KaY-1(R24Q) (FIG. 6B).

FIG. 7. CD spectra of selected CT peptides and peptide analogues synthesized.

FIG. 8. Stability time courses of CT peptides and peptide analogues in simulated gastric fluid (SGF).

FIG. 9. Stability time courses of CT peptides and peptide analogues in simulated intestinal fluid (SIF).

FIG. 10. Activation of calcitonin receptor by CT peptides and peptide analogues as determined by cAMP accumulation assay.

FIG. 11. Activation of calcitonin receptor by CT peptides and peptide analogues as determined by Ca²⁺ assay.

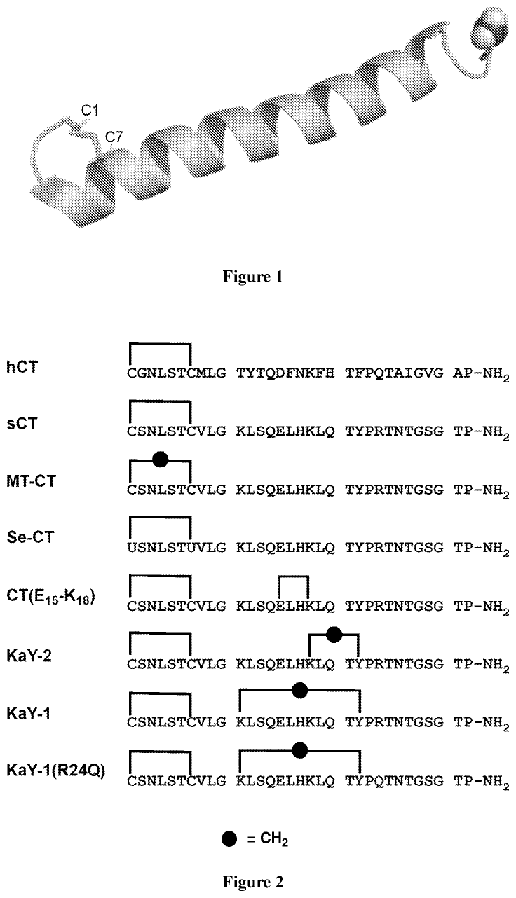

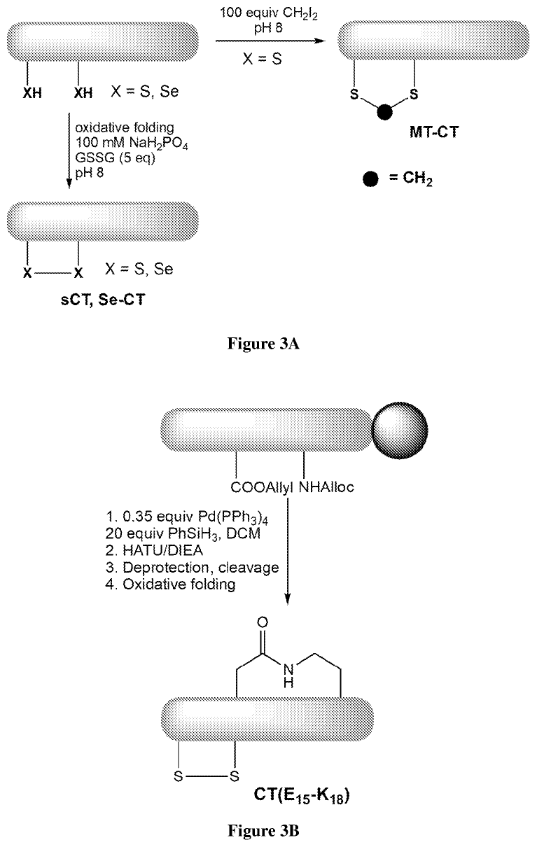

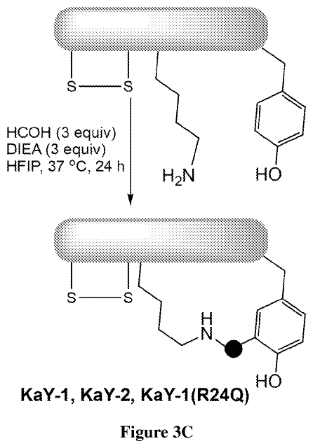

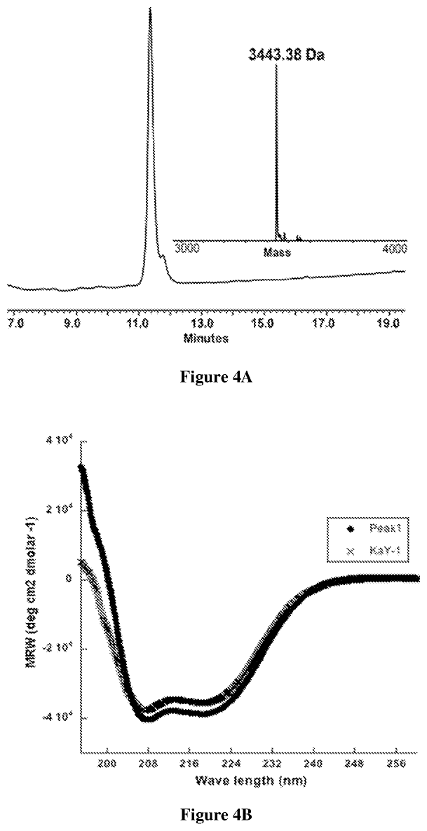

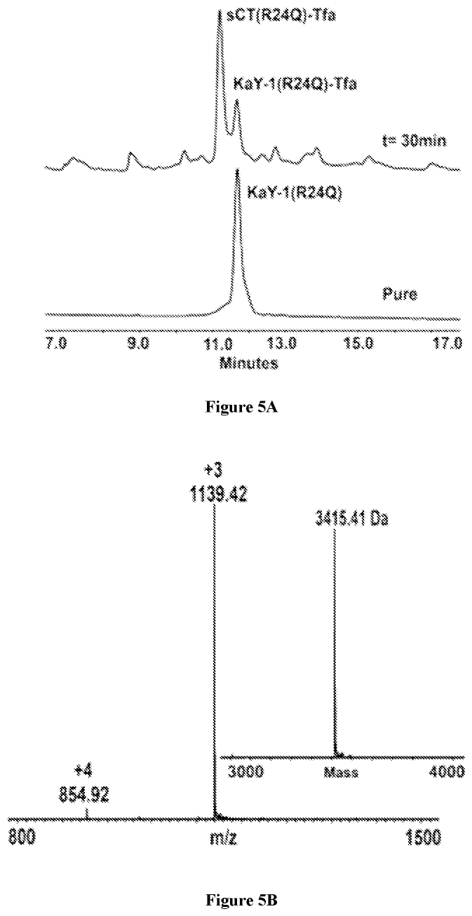

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

The present invention relates to stapled calcitonin analogues that exhibit a higher resistance under simulated gastrointestinal conditions and demonstrate activation of the calcitonin receptor. A series of analogues was synthesized, in which each analogue comprises one or more modification to the structural features of calcitonin.

Calcitonin has several conserved structural features that were considered, namely, its highly helical structure and its N-terminal disulfide bridge. Two of the analogues targeted stabilization of the disulfide bond by substituting the disulfide bridge between Cys1 and Cys7 with a methylene thioacetal (MT-CT) or a diselenide bridge (Se-CT). A third analogue applied the i, i+3 lactam stapling approach to generate a crosslink between Glu15 and Lys18. In addition, cooperative stapling between a Lys and Tyr residues with formaldehyde was used to generate a i, i+4 stapling product (between Lys18 and Tyr22) (KaY-2) and i, i+11 stapling product (between Lys11 and Tyr22) (KaY-1). Finally, using rational design, a key cleavage site for intestinal pancreatin peptidase arginine residue, R24 of sCT, was substituted with glutamine (the residue in the corresponding position in hCT) to obtain KaY-1(R24Q).

The present invention is based in part on the observation that the short elimination half-life of sCT, involving enzymatic degradation of sCT by proteolytic enzymes, could be significantly improved upon by preparing analogues with stabilizing bridges both between positions Cys1 and Cys7 as well as between Lys 11 and Tyr 22. These bridges maintain the high helicity of calcitonin needed for its activity while improving upon its stability to enzymatic degradation.

In addition, it was observed that the stability of KaY-1 could be further improved by substituting position 24 from Arg to Gln (as in hCT), thereby removing a protease site. Remarkably, KaY-1(R24Q) demonstrated a 5-fold improvement in stability in simulated gastric fluid over sCT and a 6-fold improvement in stability in simulated gastric fluid. Unexpectedly, KaY-1(R24Q) demonstrated enhanced potency in activating the cAMP pathway of the human calcitonin receptor compared with hCT. The calcitonin analogues described herein provide the basis for the development for orally-formulated calcitonin treatments.

Thus, the present invention provides according to one aspect, a stabilized, bicyclic peptide analogue of calcitonin (CT) comprising the amino acid sequence X₁-Ser-Asn-Leu-Ser-Thr-X₂-Val-Leu-Gly-Lys-Leu-Ser-Gln-Glu-Leu-His-X₃-Leu-Gln-Thr-Tyr-Pro-X₄-Thr-Asn-Thr-Gly-Ser-Gly-Thr-Pro (SEQ ID NO: 1), wherein X₁ and X₂ are independently selected from Cys, and modified Cys residues (e.g., Sec); X₃ is selected from Lys and modified Lys comprising a bulky moiety connected to its C-amine; and X₄ is selected from Gln (Q) and Arg (R), wherein a first bridge connects positions 1 and 7 and a second bridge connects positions Lys11 and Tyr22; or a derivative of SEQ ID NO: 1 comprising 1-3 modifications.

According to some embodiments, X₁ and X₂ are each a Cys residue. According to additional embodiment, the first bridge between Cys1 and Cys7 is a disulfide bridge. According to some embodiments, X₁ and X₂ in each position is a modified Cys residue independently selected from: Cys-S—H, Cys-OH, Cys-S—OH, Cys-S—O—(CH₂)₁₋₄—CH₃, Cys-S-ester-(CH₂)₁₋₄—CH₃, and Cys-S-acetamidomethyl. According to some embodiments, X₃ is Lys. According to some embodiments, X₃ is a modified Lys residue comprising a bulky moiety connected to its ε-amine. According to some embodiments, the bulky moiety is selected from a polyethylene (PEG) moiety and a fatty acid moiety.

According to some embodiments, the second bridge, between Lys11 and Tyr22, is an alkylene bridge comprising 1-6 methylene units, or a modified alkylene bridge. According to some embodiments, the second bridge is selected from a methylene bridge, an ethylene bridge, and a propylene bridge. According to some embodiments, the second bridge is a methylene bridge. According to some embodiments, the second bridge is a modified alkylene bridge. According to some embodiments, the modified alkylene bridge is selected from (—CH₂—O—CH₂—), (—CF₂—), (—CH₂CH₂—O—CH₂CH₂—), (—CF₂CF₂—) or similar linkers.

According to some embodiments, X₄ is Arg. According to some embodiments, X₄ is Gln. Each possibility represents a separate embodiment of the present invention.

The invention also provides, according to some aspects and embodiments, a pharmaceutical composition comprising one or more of the CT analogues described above, or a conjugate thereof, and at least one excipient, salt or carrier.

According to some embodiments, the pharmaceutical composition comprises at least one CT analogue selected from SEQ ID NOs: 1-4 and 11-13, a derivative thereof comprising 1-3 modifications, or a conjugate thereof.

The present invention provides according to some further aspects and embodiments, a method of treating a calcium-related or a bone-related disorder, disease or disease complication, or any disease, disorder or complication responsive to the action of calcitonin, the method comprising providing an effective amount of a stabilized bicyclic calcitonin analogue according to any one of SEQ ID NOs: 1-4 and 11-13, a derivative thereof comprising 1-3 modifications, or a conjugate thereof, to a patient in need of such a treatment. Each possibility represents a separate embodiment of the present invention.

According to some particular embodiments, the disorder is a bone disorder. According to specific embodiments, the bone disorder is a disorder caused by bone resorption including hypercalcemia, osteoporosis (including postmenopausal osteoporosis), osteolysis, Paget's disease, or a disorder characterized by arthritic conditions including rheumatoid arthritis, inflammation of the joints, swelling of skeletal joints, cartilage erosion, bone erosion, bone fractures, osteoarthritis, and bone destruction. Each possibility represents a separate embodiment of the present invention.

According to some embodiments, the disorder is selected from postmenopausal osteoporosis, Paget disease of bone, hypercalcemia, acute bone loss due to sudden immobilization and migraines. In particular embodiments, the disorder is selected postmenopausal osteoporosis, Paget disease of bone, and/or hypercalcemia. Each possibility represents a separate embodiment of the present invention.

In some embodiments, the method further comprises providing the stabilized calcitonin with at least one other treatment or medicament. According to other embodiments, the at least one other medication is selected from vitamin D, a calcium salt, bisphosphonates, loop diuretics, oral phosphate, corticosteroids, and non-steroidal anti-inflammatory drugs. Each possibility represents a separate embodiment of the present invention.

According to some embodiments, the disorder or complication is pain.

According to some embodiments, the present invention provides a method of treating or alleviating pain, the method comprising providing an effective amount of stabilized CT according to any one of SEQ ID NOs: 1-4 and 11-13. In yet other embodiments, the method comprising providing an effective amount of a derivate of stabilized CT according to any one of SEQ ID NOs: 1-4 and 11-13 comprising 1-3 modifications, or a conjugate thereof, to a patient in need of such a treatment. Each possibility represents a separate embodiment of the present invention.

Any administration route may be used in the method of treating or alleviating a disorder, disease, or complication. According to some embodiments, administration routes include parenteral or enteral routes. According to some embodiments, the method of treating or alleviating a disorder, disease or complication comprises administering the stabilized CT analogue, or a pharmaceutical composition comprising said analogue, by an administration route selected from oral and nasal. Each possibility represents a separate embodiment of the present invention.

The terms “stabilized” or “stabilized peptides” refer to peptides that are constrained by synthetic means, such as a bridge, that is formed, at least in part, from a covalent linkage between amino acids in the peptide chain. Methods of producing stabilized peptides include peptide stapling, where crosslinking the amino acid side chains or peptide backbone thereby cyclizing the peptide. Such stabilization constrains the peptide's conformation flexibility, and may lead to improvement of binding affinity, selectivity, and/or resistance to protease degradation. For example, if activity of a peptide is dependent upon the peptide adopting an alpha-helical conformation, the stabilization can constrain the peptide into maintaining its helical active conformation. Examples of crosslinking approaches include helix stabilization, such as lactam groups, disulfide mimics, hydrocarbon stapled peptides, and others. Thus, the terms “stabilized” or “stabilized peptides” are used interchangeably with “stapled”, “stapled peptides”, as well as “stitched peptides”, “crosslinking” and the like. The term “bridge” as used herein is likewise synonymous with “crosslinked” and with “cyclized”.

As used herein “peptide” indicates a sequence of amino acids linked by peptide bonds. The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins as well as selenocysteine and pyrrolysine, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanoalanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others as well known to the art.

Some of the amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their analogues are represented by one-letter codes or three-letter codes according to IUPAC conventions. When there is no indication, the L isomer was used. The D isomers are indicated by “D” or “(D)” before the residue abbreviation.

According to some embodiments of the present invention, analogues consist of 29-35, 29-33, 30-32, or 31-32 amino acids. Each possibility represents a separate embodiment of the present invention.

The term “analogue” as used herein indicates the biologically active peptide which has an amino acid sequence according to the invention except for one or more amino acid changes. The term “analogue” as used herein also covers analogues which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it and have comparable or improved activity as a wild-type or unmodified peptide. The term “derivative” may be used interchangeably with “analogue”.

According to some embodiments, an analogue according to the present invention comprises substitutions, deletions or additions of 1 to 3 amino acids. In some embodiments, an analogue has at least about 80% identity to the sequence of the peptide of the invention, for example at least about 85%, at least about 90%, at least about 93%, at least about 94%, at least about 95% identity to the sequence of the peptide of the invention.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared.

As used herein, the term “about”, when referring to a measurable value is meant to encompass variations of +/−10%, more preferably +1-5%, even more preferably +/−1%, and still more preferably +/−0.1% from the specified value.

Conservative substitutions of amino acids as known to those skilled in the art are within the scope of the present invention. Conservative amino acid substitutions include replacement of one amino acid with another having the same type of functional group or side chain e.g. aliphatic, aromatic, positively charged, negatively charged. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K), Histidine (H);
5) Isoleucine (I), Leucine (L), Methionine (M), Valine (V); and
6) Phenylalanine (F), Tyrosine (Y), Tryptophan (W)

“Salts” of the peptides of the invention are also included in its scope and are organic and inorganic salts. As used herein the term “salts” refers to both salts of carboxyl groups and to acid addition salts of amino or guanidino groups of the peptide molecule. Salts of carboxyl groups may be formed by means known in the art and include inorganic salts, for example sodium, calcium, ammonium, ferric or zinc salts, and the like, and salts with organic bases such as salts formed for example with amines such as triethanolamine, piperidine, procaine, and the like. Acid addition salts include, for example, salts with mineral acids such as, for example, acetic acid or oxalic acid.

A peptide conjugate according to the present invention comprises any peptide, peptide analogue defined above, conjugated to a carrier peptide, protein or another moiety which improves the peptide's solubility, stability or permeability. A fusion protein comprising at least one peptide according to the invention is also within this scope.

According to some embodiments the at least one peptide of the conjugate or of the fusion protein is selected from a sequence set forth in any one of SEQ ID NOs: 1-13. The present invention also provides, according to yet another aspect, a conjugate comprising a CT analogue having a sequence set forth in any one of SEQ ID NOs: 1-4 and 11-13, or a derivative thereof comprising 1-3 modifications to any of these sequences, and at least one additional moiety or sequences connected to the CT analogue or derivative. According to some embodiments, 1-15 amino acids are connected to the CT analogue or derivative to form a conjugate consisting of 30-50, 30-45, 30-40, or 30-35 amino acids. According to some embodiments, the conjugate comprises at least one non-proteinaceous moiety. According to some embodiments, the conjugate comprises one or more permeability-enhancing moieties. According to some embodiments, the conjugate comprises one or more stabilizing moieties. Each possibility represents a separate embodiment of the present invention.

According to some embodiments, the conjugate of the CT analogue comprises a permeability-enhancing moiety. Any moiety known in the art to facilitate actively or passively or enhance permeability of the compound into cells may be used in the CT analogues according to the present invention. The permeability-enhancing moiety may be connected to any position in the peptide moiety, directly or through a spacer or linker. According to some specific embodiments, the permeability-enhancing moiety is connected to the N-terminus or C-terminus of the peptide. According to other embodiments, the permeability-enhancing moiety is connected to a side chain of an amino acid residue of the peptide. According to particular embodiments, the permeability-enhancing moiety is connected to an ε-amine of a lysine residue. Each possibility represents a separate embodiment of the present invention. According to some embodiments, at least one permeability-enhancing moiety is connected through a cleavable linkage to at least one terminal or charged side chain of the peptide.

“Permeability” refers to the ability of an agent or substance to penetrate, pervade, or diffuse through a barrier, membrane, or a skin layer. A “cell permeability” or a “cell-penetration” moiety refers to any molecule known in the art which is able to facilitate or enhance penetration of molecules through membranes. Non-limitative examples of a permeability-enhancing moiety include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to some embodiments, the hydrophobic moiety is a lipid moiety or an amino acid moiety.

The optional connective spacer or linker may be of varied lengths and conformations comprising any suitable chemistry including but not limited to amine, amide, carbamate, thioether, oxyether, sulfonamide bond and the like. Non-limiting examples for such spacers include amino acids, sulfone amide derivatives, amino thiol derivatives and amino alcohol derivatives. The spacer may be used to allow distance between the permeability-enhancing moiety and the peptide.

Water soluble, synthetic polymers, particularly polyalkylene glycols, are widely used to conjugate therapeutically active molecules such as peptides. These therapeutic conjugates have been shown to alter pharmacokinetics favorably by prolonging circulation time and decreasing clearance rates, decreasing systemic toxicity, and in several cases, displaying increased clinical efficacy. The process of covalently conjugating polyethylene glycol, PEG, to proteins is commonly known as “pegylation”.

Where appropriate, the abbreviation PEG is used in combination with a numeric suffix, which indicates the number of repeating ethylene glycol units.

As used herein “PEG or derivatives thereof” refers to any compound including at least one polyethylene glycol moiety. PEGs exist in linear forms and branched forms comprising a multi-arm and/or grafted polyethylene glycols. The term “PEG derivative”, as used herein, relates to PEG which is modified by alkylation of the terminal hydroxy group. In some embodiments, the terminal hydroxyl group is alkylated by a linear or branched C1-C6 alkyl. A PEG may further comprise a functional group. A PEG may be mono-, di-, or multifunctional polyethylene glycols.

In some aspects and embodiments, CT analogues selected from SEQ ID NOs: 1, 11, and 13 are PEGylated at the ε-amine of Lys18. In some embodiments, the length of the PEG chain is between 8-20 units. In some embodiments, the length of the PEG chain is between 8-16 units. In some embodiments, the length of the PEG chain is between 10-14 units.

According to some embodiments, the CT analogue comprises at least one modification relative to any one of SEQ ID NOs: 1-4 and 11-13, said at least one modification is selected from the group consisting of: deletion, substitution, methylation or addition of amino acids, and combinations thereof. Each possibility represents a separate embodiment of the present invention.

According to some embodiments, the CT analogue comprises 1-3 modifications relative to the any of SEQ ID NOs: 2-4 and 11-13.

According to some embodiments, the C-terminus of the peptide is amidated carboxylated or esterified. According to some specific embodiments, the CT analogue comprises an amidated C-terminus. According to some embodiments, the N-terminus of the peptide is acylated or palmitoylated. According to some specific embodiments, the N-terminus is acylated. According to yet other embodiments, the N-terminal residue of the peptide is an acetylated Cys residue. In some embodiments, both the N- and C-termini are modified. Each possibility represents a separate embodiment of the present invention.

### Pharmacology

Apart from other considerations, the fact that the novel active ingredients of the invention are peptide analogues dictates that the formulation be suitable for delivery of these types of compounds. Although in general peptides are less suitable for oral administration due to susceptibility to digestion by gastric acids or intestinal enzymes, the present invention provides novel methods and designs of metabolically stable and oral bioavailable peptide analogues.

The compounds of the present invention can be formulated into various pharmaceutical forms for purposes of administration. Pharmaceutical composition of interest may comprise at least one additive selected from a disintegrating agent, binder, flavoring agent, preservative, colorant and a mixture thereof, as detailed for example in “Handbook of Pharmaceutical Excipients”; Ed. A. H. Kibbe, 3rd Ed., American Pharmaceutical Association, USA. For example, a compound of the invention, or its salt form or a stereochemically isomeric form, can be combined with a pharmaceutically acceptable carrier. Such a carrier can depend on the route of administration, such as oral, rectal, percutaneous or parenteral injection.

According to some aspects and embodiments, the present invention provides a pharmaceutical composition comprising a stabilized bicyclic calcitonin analogue according to any one of SEQ ID NOs: 1-4 and 11-13. In some embodiment, the composition comprises a derivative thereof comprising 1-3 modifications. In yet other embodiments, the composition comprises a conjugate thereof, and a pharmaceutically acceptable carrier. Each possibility represents a separate embodiment of the present invention.

According to some embodiments, the pharmaceutical composition is formulated in a form suitable for oral, nasal, intramuscular, subcutaneous or intravenous administration. According to particular embodiments, the pharmaceutical composition is formulated in a form suitable for nasal or oral administration. According to more particular embodiments, the pharmaceutical composition is formulated in a form suitable for oral administration. Each possibility represents a separate embodiment of the present invention.

A “carrier” or “excipient” as used herein refers to a non-toxic solid, semisolid or liquid filler, diluent, vehicle, solubilizing agent, encapsulating material or formulation auxiliary of any conventional type, and encompasses all of the components of the composition other than the active pharmaceutical ingredient. The carrier may contain additional agents such as wetting or emulsifying agents, or pH buffering agents. Other materials such as anti-oxidants, humectants, viscosity stabilizers, and similar agents may be added as necessary. For example, in preparing the compositions in oral dosage form, media such as water, glycols, oils, alcohols can be used in liquid preparations such as suspensions, syrups, elixirs, and solutions. Alternatively, solid carriers such as starches, sugars, kaolin, lubricants, binders, disintegrating agents can be used, for example, in powders, pills, capsules or tablets.

The pharmaceutically acceptable excipient(s) useful in the composition of the present invention are selected from but not limited to a group of excipients generally known to persons skilled in the art e.g. diluents such as lactose (Pharmatose DCL 21), starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, and bentonite; disintegrants; binders; fillers; bulking agent; organic acid(s); colorants; stabilizers; preservatives; lubricants; glidants/antiadherants; chelating agents; vehicles; bulking agents; stabilizers; preservatives; hydrophilic polymers; solubility enhancing agents such as glycerin, various grades of polyethylene oxides, transcutol and glycofiirol; tonicity adjusting agents; pH adjusting agents; antioxidants; osmotic agents; chelating agents; viscosifying agents; wetting agents; emulsifying agents; acids; sugar alcohol; reducing sugars; non-reducing sugars and the like, used either alone or in combination thereof. The disintegrants useful in the present invention include but not limited to starch or its derivatives, partially pregelatinized maize starch (Starch 1500®), croscarmellose sodium, sodium starch glycollate, clays, celluloses, alginates, pregelatinized corn starch, crospovidone, gums and the like used either alone or in combination thereof. The lubricants useful in the present invention include but not limited to talc, magnesium stearate, calcium stearate, sodium stearate, stearic acid, hydrogenated vegetable oil, glyceryl behenate, glyceryl behapate, waxes, Stearowet, boric acid, sodium benzoate, sodium acetate, sodium chloride, DL-leucine, polyethylene glycols, sodium oleate, sodium lauryl sulfate, magnesium lauryl sulfate and the like used either alone or in combination thereof. The anti-adherents or glidants useful in the present invention are selected from but not limited to a group comprising talc, corn starch, DL-leucine, sodium lauryl sulfate, and magnesium, calcium and sodium stearates, and the like or mixtures thereof. In another embodiment of the present invention, the compositions may additionally comprise an antimicrobial preservative such as benzyl alcohol. In an embodiment of the present invention, the composition may additionally comprise a conventionally known antioxidant such as ascorbyl palmitate, butylhydroxyanisole, butylhydroxytoluene, propyl gallate and/or tocopherol.

In another embodiment, the dosage form of the present invention additionally comprises at least one wetting agent(s) such as a surfactant selected from a group comprising anionic surfactants, cationic surfactants, non-ionic surfactants, zwitterionic surfactants, or mixtures thereof. The wetting agents are selected from but not limited to a group comprising oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate and the like, or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gammacyclodextrin, and hydroxypropyl beta-cyclodextrin, or the like. In yet another embodiment, the dosage form of the present invention additionally comprises of lipid(s) selected from but not limited to glyceryl behenate such as Compritol® ATO888, Compritol® ATO 5, and the like; hydrogenated vegetable oil such as hydrogenated castor oil e.g. Lubritab®; glyceryl palmitostearate such as Precirol® ATO 5 and the like, or mixtures thereof used either alone or in combination thereof. It will be appreciated that any given excipient may serve more than one function in the compositions according to the present invention. Furthermore, the term “carrier” or “excipient” includes mixtures of carriers and or excipients.

It can be advantageous to formulate the compositions of the invention in dosage unit form for ease of administration and uniformity of dosage. “Dosage unit form” refers to physically discrete units suitable as unitary dosages, each unit containing a pre-determined quantity of active ingredient calculated to produce the desired therapeutic effect in association with the chosen carrier.

Other routes of administration are intra-articular, intravenous, intramuscular, subcutaneous, topical, transdermal, intradermal, or intrathecal.

According to some embodiments, the pharmaceutical composition is for use in preventing or treating a calcium-related or a bone-related disorder, disease or disease complication or any disorder, disease or complication known to be responsive to the action of calcitonin.

“Disorders responsive to the action of calcitonin” are, e.g., various types of calcium-related or bone disorders. A first class of disorders fall in the category relating to disorders caused by bone resorption. Examples of such disorders are hypercalcemia, osteoporosis, osteolysis and Paget's disease, a chronic bone disorder that typically results in enlarged, deformed bones due to excessive breakdown and formation of bone tissue that can cause bones to weaken and may result in bone pain, arthritis, deformities or fractures. A second class of disorders is characterized by arthritic conditions. An example of such disorders is osteoarthritis. rheumatoid arthritis (RA), a chronic, systemic, inflammatory autoimmune disease, has as its primary target the synovial tissues. RA is characterized by inflammation and swelling of skeletal joints, especially the small joints of the extremities, leading to erosion and destruction of cartilage and bone. The present invention may be used to inhibit, halt or even reverse the cartilage and bone erosion, fracture and destruction, and to decrease the pain, associated with rheumatoid arthritis Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Dyestuffs or pigments may be added to the tablets or dragee coatings for identification or to characterize different combinations of active compound doses.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

For administration by inhalation, the variants for use according to the present invention can be conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. The peptide analogues of the invention may be formulated in sterile water or in physiologically compatible buffers such as Hank's solution, Ringer's solution, physiological saline buffer or glucose buffer or appropriate combinations thereof. Other ingredients may be included to aid in solubility. In addition, solid preparations that are converted to liquid form shortly before use can be made. For percutaneous administration, the carrier can include a penetration enhancing agent or a wetting agent.

Additionally, suspensions of the active compounds may be prepared as appropriate oily injection suspensions. Suitable natural or synthetic carriers are well known in the art (Pillai et al., 2001, Curr. Opin. Chem. Biol. 5, 447). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

For transmucosal administration, penetrants appropriate to the barrier to be permeated are used in the formulation. Such penetrants, for example, polyethylene glycol, are generally known in the art.

The compounds of the present invention may also be formulated in rectal compositions such as suppositories or retention enemas, using, e.g., conventional suppository bases such as cocoa butter or other glycerides.

In some embodiments, the pharmaceutical compositions of the invention are formulated for local administration. In some embodiments, the compositions may be formulated for topical, subcutaneous or transdermal administration.

According to some embodiments, the pharmaceutical composition for topical and/or transdermal administration is formulated as an ointment, cream, lotion, or spray. For example, the pharmaceutical composition may be formulated in a cream for topical application to the skin, in a powder, in a liquid, in a dry formulation, and the like. Other formulations will be readily apparent to one skilled in the art.

The peptide analogues, derivatives and conjugates of present invention may be produced by any method known in the art, including recombinant and synthetic methods and combinations thereof.

The procedures utilized to construct peptide compounds of the present invention generally rely on the known principles of peptide synthesis. However, it will be appreciated that accommodation of the procedures to the specific sequences of the present invention may be required. Examples of several synthetic procedures are given below.

Solid Phase Peptide Synthesis Solid phase peptide synthesis (SPPS) procedures are well known in the art and further described in “Solid-Phase Synthesis: A Practical Guide”, Ed. Steven A. Kates and Fernando Albericio, CRC Press; 1st Edition (2000). A skilled artesian may synthesize any of the peptides of the present invention by using an automated peptide synthesizer using standard chemistry such as, for example, t-Boc or Fmoc chemistry. The methods include exclusive solid phase synthesis, partial solid phase synthesis, fragment condensation, classical solution synthesis.

Coupling of the amino acids in solid phase peptide chemistry can be achieved by means of a coupling agent such as but not limited to dicyclohexycarbodiimide (DCC), bis(2-oxo-3-oxazolidinyl) phosphinic chloride (BOP—Cl), benzotriazolyl-N-oxytrisdimethyl-aminophosphonium hexafluoro phosphate (BOP), 1-oxo-1-chlorophospholane (Cpt-Cl), hydroxybenzotriazole (HOBT), or mixtures thereof.

The use of additional coupling reagents including, but not limited to: coupling reagents such as PyBOP (Benzotriazole-1-yl-oxy-tris-pyrrolidino-phosphonium hexafluorophosphate), PyBrOP (Bromo-tris-pyrrolidino-phosphonium hexafluoro-phosphate), HBTU (2-(1H-Benzotriazole-1-yl)-1,1,3,3-tetramethyluronium hexafluoro-phosphate), TBTU (2-(1H-Benzotriazole-1-yl)-1,1,3,3-tetramethyluronium tetrafluoroborate), may be also utilized for synthesizing the peptide compounds of the present invention.

Additional coupling chemistries may be used, such as pre-formed urethane-protected N-carboxy anhydrides (UNCA'S), pre-formed acyl halides most preferably acyl chlorides.

Such coupling may take place at room temperature and also at elevated temperatures, in solvents such as toluene, DCM (dichloromethane), DMF (dimethylformamide), DMA (dimethylacetamide), NMP (N-methyl pyrrolidinone), dioxane, tetrahydrofuran, diglyme and 1,3 dichloropropane, or mixtures of the above.

Synthetic peptides can be purified by preparative high performance liquid chromatography (HPLC) and the composition of which can be confirmed via amino acid sequencing.

The sequences of peptide analogues according to some embodiments of the present invention are provided in Table 1.

TABLE 1
Calcitonin analogues

| SEQ | | | |
|---|---|---|---|
| ID | | | |
| NO: | Name | Sequence: | Features |
| 1 | | X₁SNLSTX₂VLGKLSQELHX₃ | A first bridge between X₁ and X₂ |
| | | LQTYPX₄TNTGSGTP | and a second bridge between K11 |
| | | | and Y22; X₁ is selected from Cys |
| | | | and modified Cys; X₂ is selected |
| | | | from Cys and modified Cys; X₃ is |
| | | | selected from Lys and modified Lys |
| | | | comprising a bulky moiety |
| | | | connected to its ε-amine; and X₄ is |
| | | | selected from Q and R. |
| 2 | | CSNLSTCVLGKLSQELHKL | A disulfide bridge between Cys1 |
| | | QTYPX₄TNTGSGTP-NH₂ | and Cys7; a methylene bridge |
| | | | between K11 and Y22; and X₄ is |
| | | | selected from Q and R. |
| 3 | KaY-1 | CSNLSTCVLGKLSQELHKL | A disulfide bridge between Cys1 |
| | | QTYPRTNTGSGTP-NH₂ | and Cys7; and a methylene bridge |
| | | | between K11 and Y22. |
| 4 | KaY-1 | CSNLSTCVLGKLSQELHKL | A disulfide bridge between Cys1 |
| | (R24Q) | QTYPQTNTGSGTP-NH₂ | and Cys7; and a methylene bridge |
| | | | between K11 and Y22. |
| 5 | Salmon | CSNLSTCVLGKLSQELHKL | Oncorhynchus keta (Chum salmon |
| | CT (sCT) | QTYPRTNTGSGTP-NH₂ | or Salmo keta). |
| 6 | Human CT | CGNLSTCMLGTYTQDFNKF | Homo sapiens. |
| | (hCT) | HTFPQTAIGVGAP-NH₂ | |
| 7 | MT-CT | CSNLSTCVLGKLSQELHKL | A methylene thioacetal bridge |
| | | QTYPRTNTGSGTP-NH₂ | between Cys1 and Cys7. |
| 8 | Se-CT | USNLSTUVLGKLSQELHKL | Cys 1 and Cys 7 are both substituted |
| | | QTYPRTNTGSGTP-NH₂ | with Sec (U) forming a diselenide |
| | | | bridge between Sec1 and Sec7. |
| 9 | CT | CSNLSTCVLGKLSQELHKL | A disulfide bridge between Cys1 |
| | (E₁₅-K₁₈) | QTYPRTNTGSGTP-NH₂ | and Cys7; and a lactam bridge |
| | | | between Glu15 and Lys18. |
| 10 | KaY-2 | CSNLSTCVLGKLSQELHKL | A disulfide bridge between Cys1 |
| | | QTYPRTNTGSGTP-NH₂ | and Cys7; and a methylene bridge |
| | | | between Lys18 and Tyr22. |
| 11 | pegylated | CSNLSTCVLGKLSQELHX₃L | A disulfide bridge between Cys1 |
| | KaY-1 | QTYPQTNTGSGTP-NH₂ | and Cys7; a methylene bridge |
| | (R24Q) | | between Lys11 and Tyr22; and X₃ is |
| | | | selected from Lys or modified Lys |
| | | | comprising a bulky moiety |
| | | | connected to its ε-amine. |
| 12 | Cys- | X₁SNLSTX₂VLGKLSQELHK | A bridge between X₁ and X₂; a |
| | modified | LQTYPQTNTGSGTP-NH₂ | methylene bridge between Lys11 |
| | KaY-1 | | and Tyr22; and X₁ and X₂ are |
| | (R24Q) | | selected from Cys or modified Cys. |
| 13 | Cys- | X₁SNLSTX₂VLGKLSQELHX₃ | A bridge between X₁ and X₂; a |
| | modified | LQTYPQTNTGSGTP-NH₂ | methylene bridge between Lys11 |
| | pegylated | | and Tyr22; and X₁ and X₂ are |
| | KaY-1 | | selected from Cys and modified Cys |
| | (R24Q) | | and X₃ is selected from Lys and |
| | | | modified Lys comprising a bulky |
| | | | moiety connected to its ε-amine. |

### EXAMPLES

The following examples are presented in order to more fully illustrate several embodiments of the invention. They should, in no way be construed, however, as limiting the broad scope of the invention. One skilled in the art can readily devise many variations and modifications of the principles disclosed herein without departing from the scope of the invention.

### Materials and Methods

Peptides were prepared by using an automated peptide synthesizer. Fmoc-amino acids (1 mmol in 5 mL DMF, 4 equivalents) were activated with O-(1H-6-Chlorobenzotriazole-1-yl)-1,1,3,3-tetramethyluronium hexafluorophosphate (HCTU) (1 mmol in 5 mL DMF, 4 equivalents) and N,N-Diisopropylethylamine (DIEA) (2 mmol in 5 mL of DMF, 8 equivalents) for 5 minutes and coupled for 25 minutes, with constant shaking. The Fmoc deprotection step was carried out with 20% piperidine in DMF (two separate deprotection steps, 10 min per step), and DMF was used for washing the resin. Fmoc-Sec(4-MeBzl)-OH was coupled manually using DIC/Oxyma Pure activation method. The resulting resins were washed with DMF (3×), DCM (3×) and dried. The peptide was cleaved off the resin using a trifluoroacetic acid (TFA): triisopropylsilane (TIPS):H₂O (95:2.5:2.5) cocktail with 2 equivalents of 2,2′-Dithiobis(5-nitropyridine) (DTNP) for 2.5 hours at room temperature. The cleavage mixture was filtered, and the resin was washed with TFA. The combined solutions were concentrated by N₂ bubbling, to which cold ether was added. The precipitated crude peptides were centrifuged, ether was removed, and the crude peptides were dissolved in MeCN:water (1:1) containing 0.1% TFA and water and lyophilized.

### Example 1. Synthesis of sCT

The synthesis of sCT was carried out on Rink amide resin (0.19 mmol/g, 0.25 mmol scale) on an automated peptide synthesizer. The resin was swelled in DMF for 1 h and treated with 20% of piperidine for Fmoc deprotection. Following the SPPS, the peptide was cleaved and deprotected using TFA:TIPS:water (95:2.5:2.5) cocktail to give 400 mg crude material (46%). The peptide was oxidized by dissolving it in 20 mL phosphate buffer (100 mM, pH 8) in the presence of 5 equivalents (64 mg) of glutathione disulfide (GSSG) and then purified using reverse-phase (RP)-HPLC (C4 column) to give 80 mg sCT (20% yield). The synthesized sCT was used to prepare MT-CT, KaY-1, and KaY-2. Product mass was confirmed by electron spray ionization mass spectrometry (ESI-MS) and high-resolution mass spectrometry (HR-MS).

### Example 2. Synthesis of MT-CT

For the preparation of MT-CT in which the methylene thioacetal (MT) crosslink is located between Cys1 and Cys7, 20 mg of sCT (2 mM) dissolved in 3 mL phosphate buffer (200 mM, 6 M Guanidinium-HCl, pH 8) and tris(2-carboxyethyl)phosphine (TCEP) as reducing agent (1.5 equivalents) were added to ensure that sCT was fully reduced. After the reaction was completed (confirmed by HPLC and MS), 100 equivalents of diiodomethane (CH₂12) in dimethyl sulfoxide (DMSO) were added, and the reaction was mixed overnight at 37° C. The reaction was quenched using 0.1% TFA in water and purified by preparative RP-HPLC (XBridge C4 column, 5 μM, 19×250 mm) using a gradient of 25-50% for 55 min of solvent A in B (wherein solvent A is acetonitrile (MeCN) with 0.1% TFA, while B is water with 0.1% TFA), to give 12 mg (60% isolated yield). Product mass was confirmed by ESI-MS and HR-MS.

### Example 3. Synthesis of CT Analogue with a Diselenide Bridge (Se-CT)

The synthesis of Se-CT was carried out on Rink amide resin (0.19 mmol/g, 0.25 mmol scale) on an automated peptide synthesizer. The resin was swelled in DMF for 1 h and treated with 20% of piperidine for Fmoc deprotection, and Sec1 and Sec7 were manually coupled using 2 equivalents (166 mg) of Fmoc-Sec(4-MeBzl)-OH using 2 equivalents of Oxyma pure (71 mg) with 3 equivalents (93.3 μL) of DIC for 2 h. Subsequently, half of the peptide resin was cleaved using DTNP (2 equivalents) in the cleavage cocktail TFA:TIPS:H₂O (95%: 2.5%: 2.5%) giving 200 mg crude peptide (45%), which were purified as in Example 3 giving 50 mg (25% isolated yield). Product mass was confirmed by ESI-MS and HR-MS.

### Example 4. Synthesis of CT Analogue with a Lactam Bridge Between Glu15 and Lys18 CT(E₁₅-K₁₈)

The synthesis of CT(E₁₅-K₁₈) was carried out on Rink amide resin (0.19 mmol/g, 0.25 mmol scale) on an automated peptide synthesizer. The resin was swelled in DMF for 1 h and treated with 20% of piperidine for Fmoc deprotection. Fmoc-Glu(OAll)-OH and Fmoc-Lys(Alloc)-OH were used at positions 15 and 18, respectively (corresponding to Glu15 and Lys18).

Following synthesis, (without removing the N-terminal Fmoc group), one third of the peptide-resin was taken and the Alloc/Allyl groups were selectively removed by Pd-catalysis. For that, the resin was swelled in DCM for 30 min, then 20 equivalents of PhSiH₃ and 0.35 equivalents of Pd[PPh₃]₄ in DCM were added and mixed for 3 h. The process was repeated twice. Subsequently, the resin was washed with 0.5 M DIEA in DMF and 20 mM of sodium diethyldithiocarbamate in DMF and DCM 3 times, each time for 2 min.

Next, the lactam formation between the liberated carboxyl and amine group was performed using 5 equivalents (160 mg) Hexafluorophosphate Azabenzotriazole Tetramethyl Uronium (HATU) and 10 equivalents (110 μL) DIEA in 5 mL DMF. Reaction was repeated twice, each time for 2 h and the completion of the cyclization reaction was confirmed by ninhydrin test. Subsequently the N-terminal Fmoc was deprotected by 20% piperdine (2×5 mL) and the peptide-resin was cleaved. After lyophilization, 30 mg of crude peptide was obtained. The crude peptide was oxidized using 5 equivalents GSSG and then purified by preparative HPLC as described above to yield 10 mg (33%) of CT(E₁₅-K₁₈). Product mass was confirmed by ESI-MS and HR-MS.

### Example 5. Synthesis of KaY-1 and KaY-2

The reaction of sCT with formaldehyde, DIEA in Hexafluoroisopropanol (HFIP), results in crosslinks between the ε-amine of Lys and the phenol ring of Tyr. sCT (20 mg, 6 μmol, 1 equivalents) was dissolved in 5 mL of 1,1,1,3,3,3-HFIP, and 3 equivalents of formaldehyde (HCOH) and 3 equivalents of DIEA and the reaction mixture was stirred at 37° C. for 5 h. Then 20 μL of the mixture was taken and precipitated using ether. The precipitate was dissolved in H₂O:MeCN (containing 0.1% TFA) and analyzed by HPLC and ESI-MS. The reaction yielded two main stapled peptides referred as KaY-1 and KaY-2, with a 12 Daltons (Da) increase, in the mass (3443.38 Da) relative to sCT. The observation of two peaks of identical mass was expected, since there are two Lys residues in the sequence (Lys11 and Lys18). The mass of KaY-1 (r.t. 8.9 min) and KaY-2 (r.t. 9.2 min) was 3443.63 Da, indicated the insertion of one methylene group between Tyr and the Lys residues. Thus, the synthesis provided 1 mg of KaY-1 and 5 mg of KaY-2.

### Selective Synthesis of KaY-1

To perform a selective methylene crosslinking to form KaY-1, and to identify which of the two Lys residues is bound to Tyr22 in each of these isomers, the orthogonal protecting group trifluoro-acetyl (Tfa), which is stable under acidic conditions, was used. The synthesis of sCT(Lys18-Tfa) was carried out on Rink amide resin (0.19 mmol/g, 0.25 mmol scale) on an automated peptide synthesizer. For this synthesis, Lys18 was manually coupled using 2 equivalents Fmoc-Lys(Tfa)-OH, activated with 2 equivalents HATU and 4 equivalents DIEA, and coupled for 2 h, while Lys11 was coupled with the standard Fmoc-Lys(Boc)-OH. Following the SPPS, half of the peptide-resin was cleaved and deprotected using TFA:TIPS:water (95:2.5:2.5) cocktail to give 200 mg (23%). 100 mg of the peptide was then oxidized by dissolving it in 20 mL phosphate buffer (100 mM, pH 8) in the presence of 5 equivalents (16 mg) GSSG (for 2 hours) and then purified using RP-HPLC (C4 column) to give 30 mg of pure sCT(Lys18-Tfa). Subsequently, the peptide was reacted with formaldehyde as described above, and after reaction completion, the Tfa protecting group was removed with 100 mM phosphate buffer at pH 13. Next, the pH was lowered to 5-6 using 5 M HCl, and following purification, 5 mg KaY-1 were obtained (16% isolated yield). KaY-1 with the methylene bridge between Lys11 and Tyr22 was confirmed by co-injection of the product with peak 1 from the previous synthesis as well as by CD; product mass was confirmed by ESI-MS and HR-MS (FIGS. 4A and 4B).

### Synthesis of Analogue KaY-1(R24Q)

It is well-known that sCT is degraded in the acidic environment of the gastric fluid and by proteases in the stomach and the intestine, among these enzymes are pepsin, trypsin, chymotrypsin and elastase. It was posited that KaY-1 could be further stabilized by removing the R24 protease cleavage site. To test this possibility, the synthesis of sCT(Lys18-Tfa)(R24Q) was carried out on Rink amide resin (0.19 mmol/g, 0.25 mmol scale) on an automated peptide synthesizer, as was done in the case of sCT(Lys18-Tfa) (Example 5).

After peptide synthesis completion, half of the peptide-resin was cleaved and deprotected using TFA:TIPS:water (95:2.5:2.5) cocktail to give 280 mg (32%). Then, 100 mg of the peptide was oxidized by dissolving it in 20 mL phosphate buffer (100 mM, pH 8) in the presence of 5 equivalents (15 mg) GSSG and then purified using RP-HPLC (C4 column) to give 20 mg of pure CT (Lys18-Tfa)(R24Q). Subsequently, it was reacted with formaldehyde as described in Example 5, and after reaction completion, the Tfa protecting group was removed with 100 mM phosphate buffer at pH 13, followed by lowering the pH to 5-6 using 5 M HCl, and following purification to give 2 mg (10% isolated yield) KaY-1(R24Q). KaY1(R24Q) with the methylene bridge between Lys11 and Tyr22 was confirmed by HPLC and CD as shown in FIGS. 5A and 5B, and product mass confirmed by ESI-MS and HR-MS.

These results, together with CD, indicated that KaY-2 has a methylene crosslink between Lys18 and Tyr22.

### Example 6. Circular Dichroism Analysis

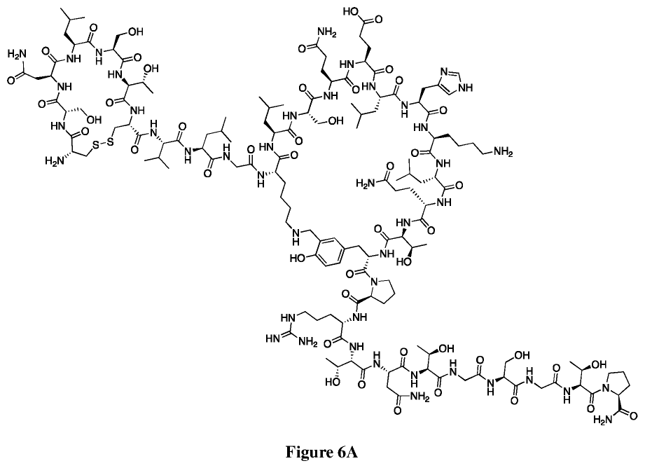

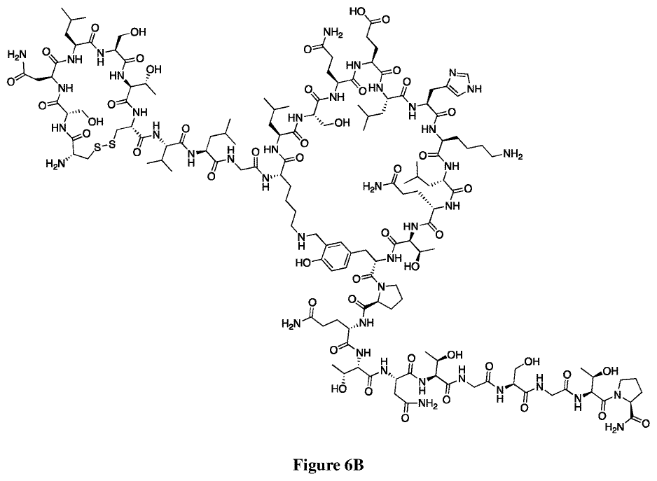

Following synthesis of the calcitonin analogues, the helicity of the calcitonin analogues was assessed by circular dichroism analysis. Far ultraviolet spectra (FIG. 6) were obtained from 195-260 nm on J-180 spectropolarimeter (Jasco) using 10-30 μM of sCT and all analogues in 10 mM phosphate buffer pH 7 and in the presence of sodium dodecyl sulphate (SDS), which were left for 2 h to allow folding. Each spectrum represents the average of 3 scans, and the mean residue ellipticity (θ_{MRE}) was calculated according to the following equation:

$${θ}_{MRE} = \frac{θ ( mdeg )}{10 \times l ( cm ) \times c ( M ) \times N ( \# amide bond )}$$

Percentage helicity of peptides as presented in Table 2 was calculated according to the following equation (Sommese, et al. 2010):

$$\% helicity = 100 \times ( \frac{∅ 222}{- 39500 \times ( 1 - \frac{2.57}{n} )} )$$

where n is the number of amino acids in the peptide.

TABLE 2
Percent α-helicity of stabilized analogues

| Calcitonin analogue | % α helicity |
|---|---|
| sCT | 98.6 |
| MT-CT | 98.5 |
| Se-CT | 99.2 |
| CT(E₁₅-K₁₈) | 62.9 |
| KaY-2 | 56.2 |
| KaY-1 | 93.7 |
| KaY-1(R24Q) | 97.4 |

The results presented in Table 2 demonstrate that analogues MT-CT, Se-CT, KaY-1 and KaY-1(R24Q) all retain the high degree of helicity following cyclization required to activate the calcitonin receptor.

### Example 7. Stability Assays of sCT and its Stabilized Analogues

### Degradation in Simulated Gastric Fluid (SGF)

sCT and its analogues were evaluated for their degradation in simulated gastric fluid (SGF). The stock solution of each analogue was prepared by dissolving 2 mg of the peptide in 1 mL water. The stock solution of pepsin enzyme was prepared by dissolving 5 mg of pepsin in 5 mL of 10 mM HCl (1 mg/mL), and then was diluted to a 0.2 mg/mL with 10 mM HCl. 7 For degradation, 34 μL of pepsin (0.2 mg/mL) was added to 100 μL of peptide (2 mg/mL) at pH 1.3 and the reaction incubated at 37° C. To monitor the degradation, 10 μL of the reaction aliquots were taken and quenched with 10 μL of 10 mM NaOH every 30 min for several hours and analyzed by HPLC using XSelect C18 column (3.5 m, 130 A, 4.6×150 mm) with a gradient of 5-70% of 0.1% TFA in MeCN over 20 min with monitoring at 220 nm.

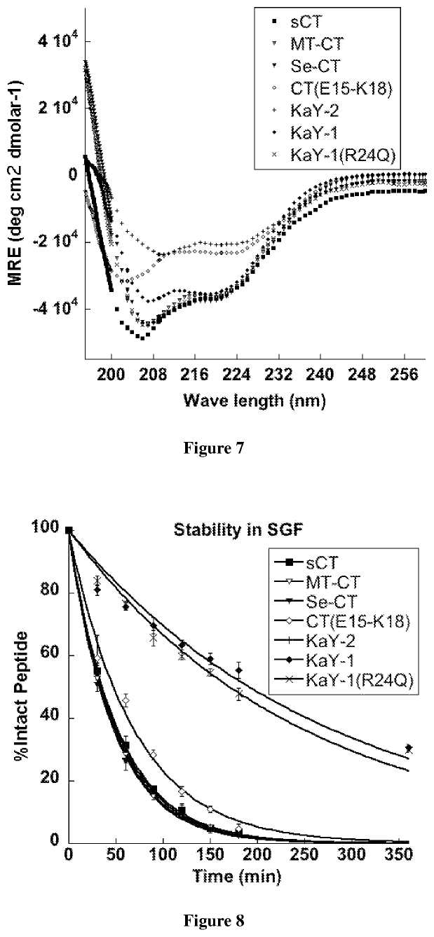

As presented in FIG. 7 and Table 3, sCT had a half-life of only 35 min in the SGF, whereas KaY-1 and KaY-1(R24Q) each had a five-fold longer half-life (190 and 170 min, respectively), while the other analogues did not exhibit any improvement in terms of stability.

Degradation of sCT, KaY-1, and KaY-1(R24Q) in Simulated Intestinal Fluid (SIF)

Based on the results for KaY-1 and KaY-1(R24Q) in SGF, the stability of these two analogues and sCT was tested in SIF. SIF was prepared by dissolving 68 mg KH₂PO₄ in 500 μL H₂O, 800 μL of 0.2 M NaOH and 10 mg porcine pancreatin. The volume was adjusted to 5 mL with water and the pH was adjusted to 6.8 to obtain 2 mg/mL pancreatin. For degradation, 3.5 μL of pancreatin (2 mg/mL) was added to 100 μL of peptide (2 mg/mL) at pH 6.8 and the reaction incubated at 37° C. To monitor degradation progress, 10 μL of the reaction aliquots were removed and quenched with 10 μL of 10 mM HCl every 30 min for several hours and quenched timepoints analyzed by HPLC using an XSelect C18 column (3.5 m, 130 A, 4.6×150 mm) with a gradient of 5-70% of 0.1% TFA in MeCN over 20 min with monitoring at 220 nm.

As presented in FIG. 8 and Table 3, KaY-1(R24Q) was found to have a t_{1/2} of 42 min, a six-fold enhancement in stability compared to sCT, while KaY-1 did not exhibit any improvements under these conditions. These findings support the proposed mechanism for CT degradation by pancreatin at Arg24, and show that by mutating this digestion site, the drug's efficacy may be increased.

### Example 8. Activation of the Calcitonin Receptor by sCT and Stabilized Analogues MT-CT, Se-CT, KaY-1 and KaY-1(R24Q)

Calcitonin receptor (CTR) is a member of the G-protein-coupled receptors superfamily. Activated CTR is capable of coupling to at least two signal transduction pathways, one of which leads to an increase in intracellular cAMP, while the other is the phospholipase C pathway (PLC) which leads to the release of Ca²⁺ from intracellular stores and promotes the influx of external calcium. Therefore, the in vitro activity of four of the most promising stabilized analogues with retained helical structures were tested in addition to synthetic sCT:MT-CT, Se-CT, KaY-1 and KaY-1(R24Q). The ability of these analogues (in the range of 10 μM-1 μM) to activate the CTR, leading to an increase in the cytosolic cAMP and Ca²⁺ concentration was evaluated.

All tested peptides were dissolved in DMSO at a final concentration of 1 mM. The working concentrations of each peptide were prepared in Opti-MEM for the cAMP assay or in the commercial kit buffer for the Ca²⁺ assay.

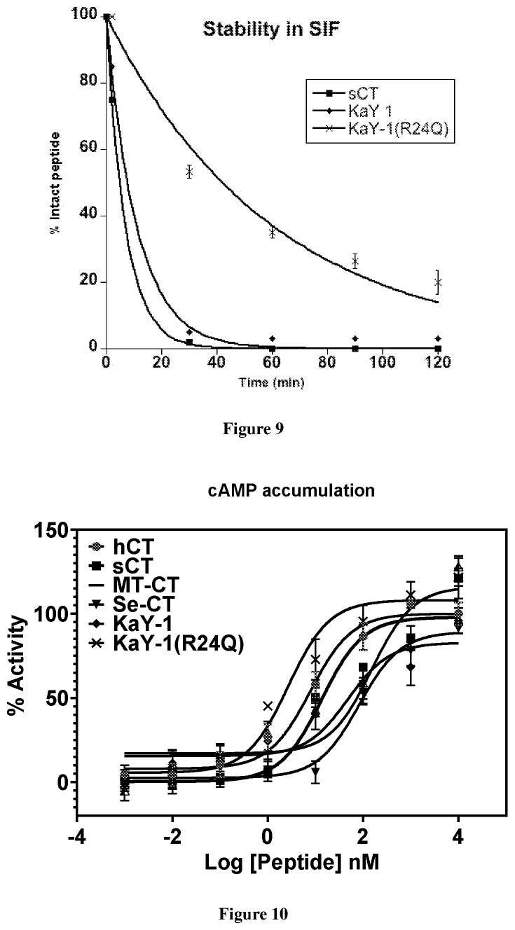

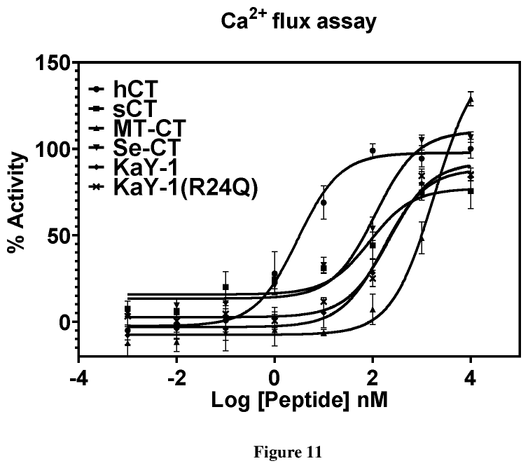

The results are depicted in FIGS. 9 and 10 and in Table 3.

cAMP Accumulation Assay

U2OS Red _{cAMP}Nomad-CALCR cell line was thawed (2×10⁶ cells per T25 flask) over the course of a day. On the second day, cells were plated into 96-well plates with a density of 20,000 cells (+/−2,000 cells) per well. Cells were maintained in DMEM-F12 medium supplemented with 10% FBS for 24 h at 37° C. in a humidified 5% CO₂ atmosphere. On day 3, the cells were incubated with the test compounds, hCT, sCT and all CT analogues, diluted in Opti-MEM overnight. Treatments were carried out in triplicate. On day 4, fluorescence intensity changes were quantified in living cells as follows:
The medium was replaced with 100 μL of PBS to perform the fluorescence intensity acquisition. For tFP650 detection, the filters were 590/20 and 665/8 nm for excitation and emission, respectively.
GraphPad Prism 9 was used for EC₅₀ calculation.

FIG. 9 shows the dose response curve for each tested peptide. As shown in Table 3, while the EC₅₀ of hCT was determined at 7.78 nM, the sCT, Se-CT, KaY-1, and KaY-1(R24Q) also exhibit EC₅₀ in the nanomolar range with KaY-1(R24Q) exhibiting unexpected enhanced potency (EC₅₀ of 2.67 nM) compared to both hCT and sCT. In contrast, MT-CT had an EC₅₀>1 μM.

### Ca²⁺ Concentration Assay

HEK293 CALCR-HiTSeeker cell line were thawed (2×10⁶ cells per T25 flask). On days 2-4, cells were maintained in DMEM supplemented with 10% FBS at 37° C. in a humidified 5% CO₂ atmosphere. On day 5, cells were plated into 96-well plates with a density of 40,000 cells (+/−2,000 cells) per well. Cells were maintained in DMEM medium supplemented with 10% FBS for 24 h at 37° C. in a humidified 5% CO₂ atmosphere. On day 7, cells were incubated with the test compounds, namely, hCT, sCT and calcitonin analogues, following the calcium kit manufacturer's instructions. GraphPad Prism 9 was used for EC₅₀ calculation.

FIG. 10 shows the dose response curve for each tested peptide. hCT showed a high efficacy (EC₅₀=2.94 nM) in activating the Ca²⁺ pathway, while in contrast MT-CT had an EC₅₀>1 μM. Gratifyingly, the EC₅₀ for sCT, KaY-1 and KaY-1(R24Q) were similar, demonstrating the therapeutic potential of the stabilizes analogues KaY-1 and KaY-1(R24Q). Table 3 further provides the values obtained with the different compounds.

TABLE 3
Summary of the t_{1/2} in SGF and SIF, and EC₅₀ for cAMP and
Ca²⁺ cytosolic concentration changes of CT analogues.

| | SGF t_{1/2} | SIF t_{1/2} | cAMP EC₅₀ | Ca²⁺ EC₅₀ |
|---|---|---|---|---|
| CT analogue | (min) | (min) | (nM) | (nM) |
| hCT | n.d. | n.d. | 7.8 ± 1.6 | 2.9 ± 0.6 |
| sCT | 35.6 ± 0.1 | 7.1 ± 0.7 | 13.5 ± 4.5 | 87.0 ± 17.5 |
| MT-CT | 33.5 ± 0.2 | n.d. | 1666 ± 60 | 7304 ± 700 |
| Se-CT | 32.6 ± 0.5 | n.d. | 82.7 ± 16.5 | 112.5 ± 18.3 |
| CT(E₁₅-K₁₈) | 47.3 ± 1.9 | n.d. | n.d. | n.d. |
| KaY-2 | 34.5 ± 0.3 | n.d. | n.d. | n.d. |
| KaY-1 | 191.2 ± 66.1 | 7.1 ± 0.7 | 47.6 ± 24.1 | 165.5 ± 34.8 |
| KaY-1(R24Q) | 170.6 ± 32.2 | 41.9 ± 6.1 | 2.7 ± 0.8 | 215.8 ± 43.8 |

### Example 10. In Vivo Assessment of Osteoporosis

In order to evaluate the pharmacological activity of the new calcitonin analogues, an animal model is established by performing ovariectomy (OVX) on female mice to induce osteoporosis, based on Hsiao, et al., 2020. Four groups of mice are used to evaluate in vivo efficacy as follow:
Group 1: Sham-OVX (healthy control),
Group 2: OVX mice treated with vehicle (negative control)
Group 3: OVX mice receiving calcitonin by injection (positive control)
Group 4: OVX mice receiving KaY-1(R24Q).

The experimental protocol is approved by the Institutional Animal Care and Use Committee of the Hebrew University of Jerusalem, which is an Association for Assessment and Accreditation of Laboratory Animal Care (AAALAC) International accredited institute.

### Dosing Regimen and Route of Administration

Mice are administered daily with Calcitonin (positive control, injected intraperitoneally), tests drugs administered orally (e.g., KaY-1(R24Q)) or Vehicle for 6 weeks.

### Sample Collection

Mice are euthanized. Once euthanized, trunk blood is collected for determining the biochemical parameters, and the femoral bones and L3 vertebrae is separated, cleaned, and fixed in 10% phosphate buffered formalin (pH 7.2) for 48 h, and then kept in 70% ethanol until further use (Baraghithy, et al., 2019A and Baraghithy et al., 2019B).

### Biochemical Analysis

To evaluate the efficacy of our candidate, bone histomorphometry (evaluation of anabolism and catabolism), micro-CT assessment (evaluation of bone mass and structure) and serum biomarkers (for bone homeostasis) are used to assess therapeutic efficacy (Kuo, et al., 2012).

### REFERENCES

Andreotti, G., Méndez, B. L., Amodeo, P., Morelli, M. A. C., Nakamuta, H., Motta, A., J. Biol. Chem., 2006, 281, 24193-24203.
Azria, M., Copp, D., Zanelli, J., Calc. Tissue Internat., 1995, 57, 405-408.
Baraghithy, S., Smoum, R., Attar-Namdar, M., Mechoulam, R., Bab, I., Tam, J., Molecules 2019, 24(20): 3719. A
Baraghithy, S., Smoum, R., Drori, A., Hadar, R., Gammal, A., Hirsch, S., et al. Journal of Bone and Mineral Research 2019, 34(1): 93-105. B
Bock, J. E., Gavenonis, J., Kritzer, J. A., ACS Chem. Biol., 2013, 8, 488-499.
Bracken, C., Gulyas, J., Taylor, J. W., Baum, J., J. Am. Chem. Soc., 1994, 116, 6431-6432.
Chang, Y. S., Graves, B., Guerlavais, V., Tovar, C., Packman, K., To, K. H., Olson, K. A., Kesavan, K., Gangurde, P., Mukherjee, A., Proc. Natl. Acad. Sci. USA, 2013, 110, E3445-E3454.
Charoenpattarapreeda, J., Tan, Y. S., Iegre, J., Walsh, S. J., Fowler, E., Eapen, R. S., Wu, Y., Sore, H. F., Verma, C. S., Itzhaki, L., Chem. Commun., 2019, 55, 7914-7917.
Chesnut III, C. H., Silverman, S., Andriano, K., Genant, H., Gimona, A., Harris, S., Kiel, D., LeBoff, M., Maricic, M., Miller, P., Amer. J. Med., 2000, 109, 267-276.
Copp, D. Ann. Rev. Physiol., 1970, 32, 61-86.
Cromm, P. M., Spiegel, J., Grossmann, T. N., ACS Chem. Biol., 2015, 10, 1362-1375.
Hamman, J. H., Enslin, G. M., Kotzé, A. F. BioDrugs, 2005, 19, 165.
Hsiao C-Y, Chen T-H, Chu T-H, Ting Y-N, Tsai P-J, Shyu J-F. Frontiers in Endocrinology 2020, 11: 613.
Iconomidou, V. A.; Leontis, A.; Hoenger, A.; Hamodrakas, S. J. FEBS Lett. 2013, 587, 569-574,
Karsdal, M., Byrjalsen, I., Henriksen, K., Riis, B., Lau, E., Arnold, M., Christiansen, C., Osteoarth. & Cartilage, 2010, 18, 150-159.
Karsdal, M., Byrjalsen, I., Alexandersen, P., Bihlet, A., Andersen, J., Riis, B., Bay-Jensen, A., Christiansen, C., Osteoarth. & Cart., 2015, 23, 532-543.
Kuo Y-J, Tsuang F-Y, Sun J-S, Lin C-H, Chen C-H, Li J-Y, et al. PLoS One 2012, 7(7): e40272.
Lau, Y. H., De Andrade, P., Quah, S. T., Rossmann, M., Laraia, L., Sköld, N., Sum, T. J., Rowling, P. J., Joseph, T. L., Verma, C., Chem. Sci., 2014, 5, 1804-1809.
Lau, Y. H., De Andrade, P., Wu, Y., Spring, D. R., Chem. Soc. Rev., 2015, 44, 91-102.
Li, B., Tang, H., Turlik, A., Wan, Z., Xue, X. S., Li, L., Yang, X., Li, J., He, G., Houk, K. N., Chen, G., Angew. Chem. Int. Ed., 2021, 60, 6646.
Li, B., Wang, L., Chen, X., Chu, X., Tang, H., Zhang, J., He, G., Li, L., Chen, G., Nature Commun., 2022, 13, 1-9.
Phelan, J. C., Skelton, N. J., Braisted, A. C., McDowell, R. S., J. Am. Chem. Soc., 1997, 119, 455-460.
Potts Jr, J. T., Bone & Mineral, 1992, 16, 169-173.
Reches, M., Porat, Y., Gazit, E., J. Biol. Chem., 2002, 277, 35475-35480.
Reginster, J. Y., Am. J. Med., 1993, 95, S44-S47.
Schafmeister, C. E., Po, J., Verdine, G. L., J. Am. Chem. Soc., 2000, 122, 5891-5892.
Sommese, R. F., Sivaramakrishnan, S., Baldwin, R. L., Spudich, J. A., Protein Science, 2010, 19 (10), 2001-2005.
Srinivasan, A., Wong, F. K., Karponis, D., J. Musculoskel. & Neur. Inter., 2020, 20, 600.
Verdine, G. L. and Walensky, L. D., Clin. Cancer Res., 2007, 13, 7264-7270.
Walensky, L. D. and Bird, G. H., J. Med. Chem., 2014, 57, 6275.
Wang, J., Yadav, V., Smart, A. L., Tajiri, S., Basit, A. W., Mol. Pharmac., 2015, 12, 966-973.
Wimalawansa, S. J., Calcified Tissue Int., 1993, 52, 90-93.
Wüster, C., Schurr, W., Scharla, S., Raue, F., Minne, H., Ziegler, R., Eur. J. Clin. Pharm., 1991, 41, 211-215.

1.-34. (canceled)

35. A stabilized, bicyclic peptide analogue of calcitonin comprising the amino acid sequence X₁-Ser-Asn-Leu-Ser-Thr-X₂-Val-Leu-Gly-Lys-Leu-Ser-Gln-Glu-Leu-His-X₃-Leu-Gln-Thr-Tyr-Pro-X₄-Thr-Asn-Thr-Gly-Ser-Gly-Thr-Pro-NH₂ (SEQ ID NO: 1), wherein X₁ and X₂ are independently selected from Cys (C) and modified Cys residues; X₃ is selected from Lys (K) and a modified Lys comprising a bulky moiety connected to its ε-amine; and X₄ is selected from Gln (Q) and Arg (R), wherein a first bridge connects positions 1 and 7 and a second bridge connects positions Lys11 and Tyr22, or a derivative thereof comprising 1-3 modifications to SEQ ID NO: 1, wherein the stabilized bicyclic calcitonin analogue consists of 29-35 amino acids.

36. The bicyclic peptide according to claim 35, wherein X₁ and X₂ are each independently selected from a Cys (C) and Sec (U) residue.

37. The bicyclic peptide according to claim 35, wherein X₁ and X₂ are each a Cys residue.

38. The bicyclic peptide according to claim 35, wherein X₃ is selected from Lys (K) and a modified Lys residue comprising a bulky moiety connected to its ε-amine.

39. The bicyclic peptide according to claim 38, wherein the bulky moiety is selected from a polyethylene glycol (PEG) moiety comprising 8-20 units (PEG₈₋₂₀) and a fatty acid moiety.

40. The bicyclic peptide according to claim 35, wherein X₄ is selected from Arg (R) and Gln (Q).

41. The bicyclic peptide according to claim 35, wherein the first bridge between Cys1 and Cys7 is a disulfide bridge and wherein the second bridge between Lys11 and Tyr22 is an alkylene bridge comprising 1-6 methylene units, or a modified alkylene bridge.

42. The bicyclic peptide according to claim 35, wherein the 1-3 modifications are selected from substitutions, additions, or deletions of amino acids, modification of at least one of the terminals of the peptide, and combinations thereof.

43. The bicyclic peptide according to claim 35, wherein the N-terminus of the peptide is acetylated and/or wherein the C-terminus is amidated.

44. The bicyclic peptide according to claim 35 comprising the amino acid sequence Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-X₄-Thr-Asn-Thr-Gly-Ser-Gly-Thr-Pro-NH₂ (SEQ ID NO: 2), wherein X₄ is selected from Q and R and wherein a disulfide bridge connects residues Cys1 and Cys7, and a methylene bridge connects residues Lys11 and Tyr22.

45. The bicyclic peptide according to claim 35 consisting of a sequence selected from one of the following:
Cys-Ser-Asn-Leu-Ser-Thr-Cys-Val-Leu-Gly-Lys-Leu-Ser-Gln-Glu-Leu-His-X₃-Leu-Gln-Thr-Tyr-Pro-Gln-Thr-Asn-Thr-Gly-Ser-Gly-Thr-Pro-NH₂, wherein a disulfide bridge connects residues Cys1 and Cys7, a methylene bridge connects residues Lys11 and Tyr22, and X₃ is selected from Lys or modified Lys comprising a bulky moiety connected to its ε-amine (SEQ ID NO: 11);
X₁-Ser-Asn-Leu-Ser-Thr-X₇-Val-Leu-Gly-Lys-Leu-Ser-Gln-Glu-Leu-His-Lys-Leu-Gln-Thr-Tyr-Pro-Gln-Thr-Asn-Thr-Gly-Ser-Gly-Thr-Pro-NH₂, wherein X₁ and X₂ are independently selected from Cys (C) and modified Cys residues and wherein a bridge connects residues X₁ and X₂, and a methylene bridge connects residues Lys11 and Tyr22 (SEQ ID NO: 12);
X₁-Ser-Asn-Leu-Ser-Thr-X₇-Val-Leu-Gly-Lys-Leu-Ser-Gln-Glu-Leu-His-X₃-Leu-Gln-Thr-Tyr-Pro-Gln-Thr-Asn-Thr-Gly-Ser-Gly-Thr-Pro-NH₂, wherein X₁ and X₂ are independently selected from Cys (C) and modified Cys residues and wherein a bridge connects residues X₁ and X₂, a methylene bridge connects residues Lys11 and Tyr22, and X₃ is selected from Lys or modified Lys comprising a bulky moiety connected to its ε-amine (SEQ ID NO: 13).

46. A conjugate of 30-50 amino acids comprising the stabilized bicyclic calcitonin analogue according to claim 35.

47. A pharmaceutical composition comprising the stabilized bicyclic calcitonin analogue according to claim 35 or a conjugate thereof, and at least one excipient, salt or carrier.

48. The pharmaceutical composition according to claim 47, wherein the stabilized bicyclic calcitonin analogue is selected from SEQ ID NOs: 1-4 and 11-13 and a derivative thereof comprising 1-3 modifications to any of said sequences.

49. A method of treating a calcium-related or a bone-related disorder, disease or disease complication, or any disease, disorder or complication responsive to the action of calcitonin, the method comprising administering to a subject in need of such a treatment a therapeutically effective amount of the stabilized bicyclic calcitonin analogue according to claim 35 or a conjugate thereof.

50. The method according to claim 49, wherein the method comprises administration by a route selected from oral, nasal, intramuscular, subcutaneous, and intravenous.

51. The method according to claim 49, wherein the stabilized bicyclic calcitonin analogue is selected from SEQ ID NOs: 1-4 and 11-13, and a derivative thereof comprising 1-3 modifications.

52. The method according to claim 49, wherein the calcium-related or the bone-related disorder, disease or disease complication is selected from a bone disorder caused by bone resorption, a disorder characterized by arthritic conditions, and pain.

53. The method according to claim 49 further comprising providing the stabilized bicyclic calcitonin analogue with at least one other treatment or medicament.

54. The method according to claim 53, wherein the at least one other medication is selected from vitamin D, a calcium salt, bisphosphonates, loop diuretics, oral phosphate, corticosteroids, and non-steroidal anti-inflammatory drugs.
